SEQUENCE LISTING (2 sequences)

SEQ ID NO: 1 - protein, 119 aa, Mus musculus
EVQLQQSGAELVKPGASVKLSCTASGFNIKDTYMHWVKQRPEQGLEWIGRIDPANGNTKYDPKFQGKATIIADTSSNTAYLQLSSLTSEDTAVYYCARGVYHDYDGDYWGQGTTLTVSS

SEQ ID NO: 2 - protein, 107 aa, Mus musculus
DIVMTQSQKFMSTSVGDRVSVTCKASQNVGTNVAWYQQKPGQSPKALIYSTSYRYSGVPDRFTGSGSGTDFTLTISNVQSEDLAEYFCQQYNSYPYTFGGGTKLEIK

(19) United States
(12) Patent Application Publication
(10) Pub. No.: US 2012/0114555 A1
(43) Pub. Date: May 10, 2012

(54) METHOD FOR TREATING IMMUNE DISORDERS

(75) Inventors: Andrew T. Hutchinson, New South Wales (AU); Darren R. Jones, New South Wales (AU); Robert L. Raison, New South Wales (AU); Parisa Asvadi, New South Wales (AU); Rosanne D. Dunn, New South Wales (AU)

(73) Assignee: IMMUNE SYSTEM THERAPEUTICS LTD, New South Wales (AU)

(21) Appl. No.: 13/263,043

(22) PCT Filed: Apr. 7, 2010
(86) PCT No.: PCT/AU2010/000394

§ 371 Date: Jan. 24, 2012

Related U.S. Application Data
(62) Division of application No. 61/167,497, filed on Apr. 7, 2009.

(51) Int. Cl. A61K 51/10; A61K 49/00; A61P 37/06; A61P 19/02; A61P 25/00; A61K 39/395; A61P 3/10

(52) U.S. Cl. 424/1.49; 424/173.1; 424/178.1; 424/85.1; 424/85.4; 424/9.1

(57) ABSTRACT

The present invention relates to the treatment of B-cell mediated immune disorders such as rheumatoid arthritis, systemic lupus erythromatomus, diabetes mellitus, and multiple sclerosis. In particular, the present invention relates to the treatment of B-cell mediated immune disorders using antibodies which bind to membrane-bound free light chain expressed on the surface of plasma cell precursors.

### FIELD OF INVENTION

The present invention relates to the treatment of B-cell mediated immune disorders such as autoimmune diseases, inflammatory disorders and sepsis. In particular, the present invention relates to the treatment of B-cell mediated immune disorders using antibodies which bind membrane-bound free light chain.

### BACKGROUND OF THE INVENTION

The ability of the immune system to distinguish ‘self’ from ‘non-self’ components is crucial, as the aberrant recognition of self components results in damaged tissues and autoimmune diseases such as rheumatoid arthritis, systemic lupus erythematosus, Sjogren's syndrome and multiple sclerosis (Browning, 2006).

Although T-cells have been the focus of autoimmune disease research for decades, the development of the B-cell-depleting antibody rituximab as a lymphoma therapy has provided a tool for the investigation of the role of B cells in immune disorders (Browning, 2006). Rituximab is a potent B-cell-cytolytic chimeric IgG1 CD20 specific monoclonal antibody which kills B-cells mainly by antibody-dependent cell-mediated cytotoxicity (ADCC). The target for Rituximab is CD20, which is expressed from the pre-B-cell stage to the pre-plasma cell stage (Edwards and Cambridge, 2006). Following injection of anti-CD20 antibody, antibody-coated B-cells in the periphery are rapidly depleted to very low levels and remain at low levels for about 6 to 12 months (Browning, 2006).

Repeated cycles of B-cell depletion with Rituximab can be associated with a decrease in total immunoglobulins, for IgG as low as 3.5 G L⁻¹ and for IgM to undetectable levels, which may limit the number of times B-cell depletion can be carried out (Edwards and Cambridge, 2006).

There remains a need for new methods for treating B-cell mediated immune disorders.

### SUMMARY OF THE INVENTION

The present inventors have now identified that membrane-bound free light chain (mFLC) is expressed on plasma cell precursors. mFLC on plasma cell precursors provides a target for the treatment or prevention of B-cell mediated immune disorders.

The therapeutic methods proposed by the present inventors are based on the administration of an antibody which binds specifically to mFLC for the depletion of plasma cell precursors in a subject suffering from a B-cell mediated immune disorder. One advantage of this therapeutic target is that it is not present on normal B cells.

Accordingly, the present invention provides a method for the treatment or prophylaxis of a B-cell mediated immune disorder in a subject, the method comprising administering to the subject an effective amount of an antibody which binds membrane-bound free light chain (mFLC).

In a preferred embodiment, the antibody binds mFLC on a plasma cell precursor, for example, a plasmablast.

In one embodiment, the plasmablast is positive for the markers CD27, CD38 and CD45. In one particular embodiment, the plasmablast is negative for the marker IgD.

In another preferred embodiment, the antibody binds specifically to mFLC.

In one embodiment, the antibody which is administered to the subject is conjugated to a cytotoxic moiety or biological response modifier. By way of non-limiting examples, the cytotoxic moiety may be a cytotoxic drug or an enzymatically active toxin of bacterial or plant origin (such as gelonin), or an enzymatically active fragment (“A chain”) of such a toxin. Enzymatically active toxins and fragments thereof are preferred and are exemplified by gelonin, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytoiacca americana proteins (PAPI, PAPII, and PAP-S), momordica charantia inhibitor, curcin, crotin, saponaria officinalis inhibitor, mitogellin, restrictocin, phenomycin, and enomycin. The cytotoxic moiety may also be a radioactive agent. In particular, the cytotoxic moiety may be a radionuclide such as, for example, Yttrium-90 (⁹⁰Y), Indium-111 (¹¹¹In), Iodine-131 (¹³¹I) or copper-67 (⁶⁷Cu). Preferably, the cytotoxic moiety is a toxin, a chemotherapeutic agent, or a radioactive agent.

Biological response modifiers which may be coupled to the antibody which binds mFLC and used in the present invention include, but are not limited to, lymphokines and cytokines such as IL-2 and interferons (α, β or γ). These biological response modifiers have a variety of effects on cells. Among these effects are increased cell killing by direct action as well as increased cell killing by increased host defence mediated processes. Conjugation of antibody which binds mFLC to these biological response modifiers will allow selective localization within plasma cell precursors, hence suppressing non-specific effects leading to toxicity of non-target cells. The biological response modifier is preferably a lymphokine, a cytokine or an interferon.

In another embodiment, the cytotoxic moiety is a nucleic acid molecule encoding a cytotoxic polypeptide.

While any suitable antibody which binds mFLC may be used in the method of the present invention, in one embodiment the antibody binds KMA or LMA. In one particular embodiment, the antibody binds KMA.

In one embodiment, the antibody which binds mFLC is a K121-like antibody. Preferably, the K121-like antibody comprises the VH region set forth in SEQ ID NO:1 and the VL region set forth in SEQ ID NO:2 or competes with an antibody having the VH region set forth in SEQ ID NO:1 and the VL region set forth in SEQ ID NO:2 for binding to kappa myeloma antigen (KMA).

In one embodiment, the B-cell mediated disorder that is treated by the method of the invention is an autoimmune disease. In one particular embodiment, the autoimmune disease is selected from rheumatoid arthritis, systemic lupus erythromatomus, diabetes mellitus, and multiple sclerosis.

The present invention further provides a method of inhibiting the growth of or killing plasma cell precursors in a subject, the method comprising administering to the subject an effective amount of an antibody which binds mFLC. By way of example, the inhibition or killing of the plasma cell precursors may be effected by apoptosis or by the immune cells of the subject (such as by antibody-dependent cell-mediated cytotoxicity (ADCC)), and/or by antibody-dependent cellular phagocytosis

The present invention also provides a method for inhibiting or killing plasma cell precursors in a subject by administering an antibody which binds mFLC which is conjugated with a cytotoxic moiety or biological modifier.

Preferably, the cytotoxic moiety is a toxin, a chemotherapeutic agent, or a radioactive agent.

In one embodiment, the cytotoxic moiety is a nucleic acid molecule encoding a cytotoxic polypeptide.

In another embodiment, the biological response modifier is a lymphokine, a cytokine or an interferon.

The present invention further provides a method for localizing plasma cell precursors in a subject, the method comprising administering to the subject an antibody which binds mFLC, allowing the antibody to bind to cells within the subject, and determining the location of the antibody within the subject.

In one embodiment, the antibody is detectably labeled.

Preferably, the antibody used in the methods of the present invention is a chimeric antibody or a humanised antibody.

The present invention further provides use of an antibody which binds mFLC for the manufacture of a medicament for the treatment of a B-cell mediated immune disorder.

The present invention further provides a method for autologous hematopoietic cell transplantation in a subject, the method comprising

(i) removing a hematopoietic progenitor cell population from the subject,

(ii) treating the cell population with an antibody which binds mFLC, and

(iii) transplanting the treated cell population from step (ii) into the subject.

The step of treating the progenitor cell population with an antibody which binds mFLC preferably involves contacting the cell population with an antibody which binds mFLC under conditions sufficient for the binding of the antibody to plasma cell precursors present in the population to inhibit the growth of, or to kill, the plasma cell precursors.

In one embodiment, the method further comprises intravenous infusion of an antibody which binds mFLC into the subject.

In yet another embodiment, the method of autologous transplantation is performed on the subject during or after cytoreductive therapy.

In yet a further preferred embodiment, the antibody which binds mFLC is bound to a solid support.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE FIGURES

FIG. 1. KMA expression on IL-21, anti-CD40 and anti-IgM activated B cells (CD 19+). Peripheral B cells were activated in the presence of IL-21, anti-CD40 and anti-IgM. At Day 6, cells were harvested then stained for KMA, IgD, CD27 and CD38. Gate 1.=CD38low, Gate 2.=CD38+ and Gate 3.=CD38++.

FIG. 2. KMA expression on SAC activated plasmablasts. Peripheral B cells were activated in the presence of IL-21, anti-CD40 and anti-IgM. At Day 6, cells were harvested then stained for KMA, IgD, CD27 and CD38. Gate 1.=CD38low, Gate 2.=CD38+ and Gate 3.=CD38++.

FIG. 3. KMA is expressed on CD38++CD45+ immature plasma cells. Tonsillar derived mononuclear cells were stained for KMA, CD19, CD38, CD45 and SYTOX Green. (a) Viable B cells were gated according to CD19 and lack of SYTOX green staining. Cells were then gated according to CD38 expression Gate 1.=CD38low, Gate 2.=CD38+ and Gate 3.=CD38++. (b) Cells were stained for KMA, CD38, CD45 and SYTOX green. Viable cells were gated according to CD38++ expression and analyzed for KMA and CD45 positivity.

FIG. 4. Identification of KMA expressing cells with plasmablast and plasma cell morphology by IHC. Unfixed sections from normal tissue were stained with FITC labeled MDX1097 followed by mouse anti-FITC secondary then anti-mouse peroxidase. Sections were developed with chromagen substrate. Panel 1=tonsillar section, panel 2=salivary gland and panel 3=peripheral blood. Arrows represent KMA+ cells.

FIG. 5. KMA is expressed on IL-21, anti-CD40 and anti-IgM activated plasmablasts. Peripheral B cells were activated in the presence of IL-21, anti-CD40 and anti-IgM. At Day 6 cells were harvested and stained for KMA, CD27, CD38, CD45 and surface IgD (not shown). Panel (a) is the scatter profile of the harvested cells. All subsequent analyses were performed in the live gate (gate 2). Panel (b) shows the CD27 and CD38 profile of the gated population. Panel (c) shows the percent cells positive for KMA (in comparison with the isotype control) within the populations coloured in Panel (b). gate 3=CD38+/−CD27−, gate 4=CD38+/−CD27+ and gate 5=CD38++CD27++. KMA was detected using APC labeled MDX-1097, and the isotype control was an APC labeled human IgG1 antibody. Results shown are representative of 5 independent experiments.

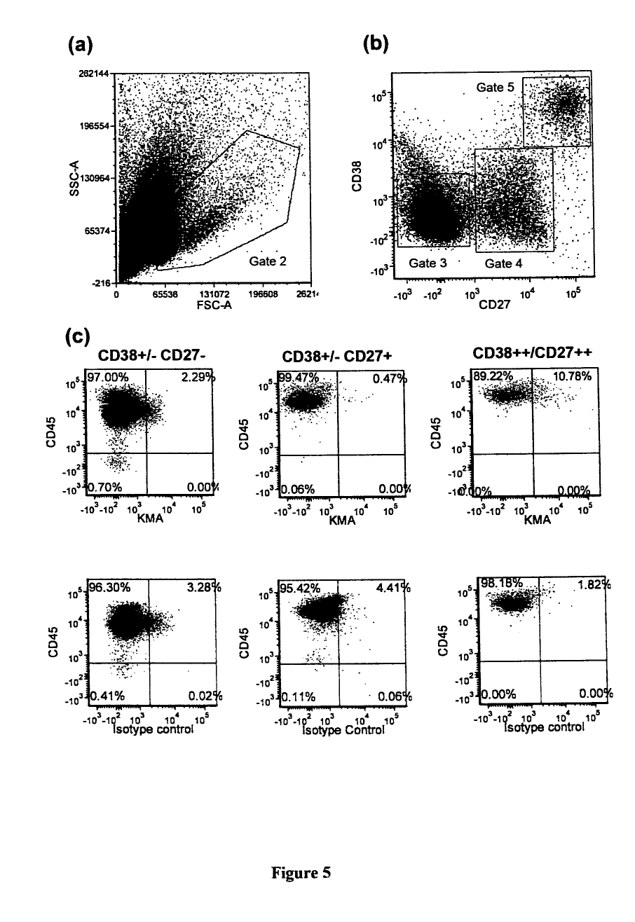

FIG. 6. LMA is expressed on IL-21, anti-CD40 and anti-IgM activated plasmablasts. Peripheral B cells were activated in the presence of IL-21, anti-CD40 and anti-IgM. At Day 6 cells were harvested and stained for LMA, CD27, CD38, CD45 and surface IgD (not shown). Panel (a) is the scatter profile of the harvested cells. All subsequent analyses were performed in the live gate (gate 1). Panel (b) shows the CD27 and CD38 profile of the gated population. Panel (c) shows the percent cells positive for LMA (in comparison with the isotype control) within the populations coloured in Panel (b). gate 2=CD38+/−CD27−, gate 3=CD38+/−CD27+ and gate 4=CD38++CD27++. LMA was detected using APC labeled 4G7, and the isotype control was an APC labeled mouse IgG1 antibody. Results shown are representative of 5 independent experiments

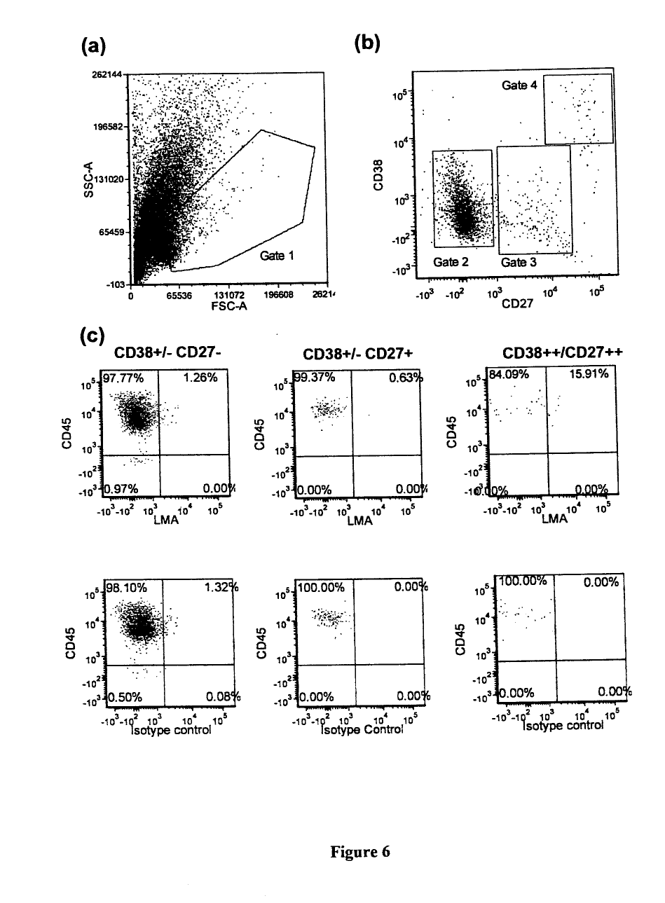

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (e.g., in immunology, immunohistochemistry, cell culture, molecular genetics, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3^{rd} edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

By “membrane-bound free light chain” is meant membrane-bound light chain that is not associated with an intact immunoglobulin. The membrane-bound light chain may comprise membrane-bound kappa light chains and/or membrane-bound lambda light chains.

As used herein, the term “plasma cell precursor” refers to cells having characteristics similar to those of activated, proliferating precursors of long lived antibody secreting plasma cells. Plasma cell precursors include pro B-cells, pre-B-cells, immature B-cells, naïve B-cells, naïve activated B-cells, germinal centre B-cells, post germinal centre B-cells, memory B-cells, and plasmablasts.

When we refer to a cell as being “positive” for a given marker it may be either a low (+, lo or dim) or a high (++, bright, bri) expresser of that marker depending on the degree to which the marker is present on the cell surface, where the terms relate to intensity of fluorescence or other colour used in the colour sorting process of the cells. The distinction of low and high will be understood in the context of the marker used on a particular cell population being sorted. When we refer herein to a cell as being “negative” for a given marker, it does not mean that the marker is not expressed at all by that cell. It means that the marker may be expressed at a relatively very low level by that cell, and that it generates a very low signal when detectably labelled.

As used herein, the terms “treating”, “treat” or “treatment” include administering a therapeutically effective amount of an antibody as described herein sufficient to reduce or delay the onset or progression of a B-cell mediated immune disorder, or to reduce or eliminate at least one symptom of a B-cell mediated immune disorder.

Antibodies which Bind Membrane-Bound Free Light Chain

The present inventors have now shown, for the first time, that membrane-bound free light chain is expressed on the surface of plasma cell precursors. Antibodies directed against mFLC will be capable of killing plasma cell precursors through mechanisms such as ADCC, complement dependent lysis, antibody-dependent cellular phagocytosis, and apoptosis and will therefore be effective therapeutic agents against B-cell mediated immune disorders. In addition, antibodies directed against mFLC can be used to deliver cytotoxins directly to plasma cell precursors.

Although not essential, the antibody may bind specifically to mFLC. The phrase “binds specifically” means that under particular conditions, the antibody binds mFLC and does not bind to a significant amount to other proteins or carbohydrates. It is preferred that an antibody that binds specifically to mFLC does not bind light chain associated with intact immunoglobulin. Specific binding to mFLC under such conditions may require an antibody that is selected for its specificity. A variety of immunoassay formats may be used to select antibodies specifically immunoreactive with mFLC. For example, solid-phase ELISA immunoassays are routinely used to select antibodies specifically immunoreactive with a protein or carbohydrate. See Harlow and Lane (1988) Antibodies, a Laboratory Manual, Cold Spring Harbor Publications, New York, for a description of immunoassay formats and conditions that can be used to determine specific immunoreactivity.

The term “antibody” as used herein includes polyclonal antibodies, monoclonal antibodies, bispecific antibodies, diabodies, triabodies, heteroconjugate antibodies, chimeric antibodies including intact molecules as well as fragments thereof, and other antibody-like molecules. Antibodies include modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light (VL) and heavy chain (VH) variable regions which may be joined directly or through a linker, or Fd fragments containing the heavy chain variable region and the CH1 domain. A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)₂ and FabFc₂ fragments which contain the variable regions and parts of the constant regions. Complementarity determining region (CDR)-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric (Morrison et al., 1984) or humanized (Jones et al., 1986), and published UK patent application No. 8707252. As used herein the term “antibody” includes these various forms. Using the guidelines provided herein and those methods well known to those skilled in the art which are described in the references cited above and in such publications as Harlow & Lane, Antibodies: a Laboratory Manual, Cold Spring Harbor Laboratory, (1988) the antibodies for use in the methods of the present invention can be readily made.

The mFLC-binding antibodies may be Fv regions comprising a variable light (V_{L}) and a variable heavy (V_{H}) chain in which the light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

### Monoclonal Antibodies

Monoclonal antibodies directed against mFLC epitopes can be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against mFLC epitopes can be screened for various properties; i.e. for isotype and epitope affinity.

Animal-derived monoclonal antibodies can be used for both direct in vivo and extracorporeal immunotherapy. However, it has been observed that when, for example, mouse-derived monoclonal antibodies are used in humans as therapeutic agents, the patient produces human anti-mouse antibodies. Thus, animal-derived monoclonal antibodies are not preferred for therapy, especially for long term use. With established genetic engineering techniques it is possible, however, to create chimeric or humanized antibodies that have animal-derived and human-derived portions. The animal can be, for example, a mouse or other rodent such as a rat.

If the variable region of the chimeric antibody is, for example, mouse-derived while the constant region is human-derived, the chimeric antibody will generally be less immunogenic than a “pure” mouse-derived monoclonal antibody. These chimeric antibodies would likely be more suited for therapeutic use, should it turn out that “pure” mouse-derived antibodies are unsuitable.

Methodologies for generating chimeric antibodies are available to those in the art. For example, the light and heavy chains can be expressed separately, using, for example, immunoglobulin light chain and immunoglobulin heavy chains in separate plasmids. These can then be purified and assembled in vitro into complete antibodies; methodologies for accomplishing such assembly have been described (see, for example, Sun et al., 1986). Such a DNA construct may comprise DNA encoding functionally rearranged genes for the variable region of a light or heavy chain of an anti-mFLC antibody linked to DNA encoding a human constant region. Lymphoid cells such as myelomas or hybridomas transfected with the DNA constructs for light and heavy chain can express and assemble the antibody chains.

In vitro reaction parameters for the formation of IgG antibodies from reduced isolated light and heavy chains have also been described (see, for example, Beychok, 1979). Co-expression of light and heavy chains in the same cells to achieve intracellular association and linkage of heavy and light chains into complete H2L2 IgG antibodies is also possible. Such co-expression can be accomplished using either the same or different plasmids in the same host cell.

### Humanising Methodologies/Techniques

In another preferred embodiment of the present invention the anti-mFLC antibody is humanized, that is, an antibody produced by molecular modeling techniques wherein the human content of the antibody is maximised while causing little or no loss of binding affinity attributable to the variable region of, for example, a parental rat, rabbit or murine antibody.

An antibody may be humanized by grafting the desired CDRs onto a human framework according to EP-A-0239400. A DNA sequence encoding the desired reshaped antibody can therefore be made beginning with the human DNA whose CDRs it is wished to reshape. The animal-derived variable domain amino acid sequence containing the desired CDRs is compared to that of the chosen human antibody variable domain sequence. The residues in the human variable domain are marked that need to be changed to the corresponding residue in the animal to make the human variable region incorporate the animal-derived CDRs. There may also be residues that need substituting in, adding to or deleting from the human sequence.

Oligonucleotides are synthesized that can be used to mutagenize the human variable domain framework to contain the desired residues. Those oligonucleotides can be of any convenient size. One is normally only limited in length by the capabilities of the particular synthesizer one has available. The method of oligonucleotide-directed in vitro mutagenesis is well known.

Alternatively, humanisation may be achieved using the recombinant polymerase chain reaction (PCR) methodology of WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

Following the mutagenesis reactions to reshape the antibody, the mutagenised DNAs can be linked to an appropriate DNA encoding a light or heavy chain constant region, cloned into an expression vector, and transfected into host cells, preferably mammalian cells. These steps can be carried out in routine fashion. A reshaped antibody may therefore be prepared by a process comprising:

(a) preparing a first replicable expression vector including a suitable promoter operably linked to a DNA sequence which encodes at least a variable domain of an Ig heavy or light chain, the variable domain comprising framework regions from a human antibody and the CDRs required for the humanized antibody of the invention;

(b) preparing a second replicable expression vector including a suitable promoter operably linked to a DNA sequence which encodes at least the variable domain of a complementary Ig light or heavy chain respectively;

(c) transforming a cell line with the first or both prepared vectors; and

(d) culturing said transformed cell line to produce said altered antibody.

Preferably the DNA sequence in step (a) encodes both the variable domain and each constant domain of the human antibody chain. The humanized antibody can be prepared using any suitable recombinant expression system. The cell line which is transformed to produce the altered antibody may be a Chinese Hamster Ovary (CHO) cell line or an immortalised mammalian cell line, which is advantageously of lymphoid origin, such as a myeloma, hybridoma, trioma or quadroma cell line. The cell line may also comprise a normal lymphoid cell, such as a B-cell, which has been immortalised by transformation with a virus, such as the Epstein-Barr virus. Most preferably, the immortalised cell line is a myeloma cell line or a derivative thereof.

The CHO cells used for expression of the antibodies may be dihydrofolate reductase (dhfr) deficient and so dependent on thymidine and hypoxanthine for growth (Urlaub and Chasin, 1980). The parental dhfr⁻ CHO cell line is transfected with the DNA encoding the antibody and dhfr gene which enables selection of CHO cell transformants of dhfr positive phenotype. Selection is carried out by culturing the colonies on media devoid of thymidine and hypoxanthine, the absence of which prevents untransformed cells from growing and transformed cells from resalvaging the folate pathway and thus bypassing the selection system. These transformants usually express low levels of the DNA of interest by virtue of co-integration of transfected DNA of interest and DNA encoding dhfr. The expression levels of the DNA encoding the antibody may be increased by amplification using methotrexate (MTX). This drug is a direct inhibitor of the enzyme dhfr and allows isolation of resistant colonies which amplify their dhfr gene copy number sufficiently to survive under these conditions. Since the DNA sequences encoding dhfr and the antibody are closely linked in the original transformants, there is usually concomitant amplification, and therefore increased expression of the desired antibody.

Another preferred expression system for use with CHO or myeloma cells is the glutamine synthetase (GS) amplification system described in WO 87/04462. This system involves the transfection of a cell with DNA encoding the enzyme GS and with DNA encoding the desired antibody. Cells are then selected which grow in glutamine free medium and can thus be assumed to have integrated the DNA encoding GS. These selected clones are then subjected to inhibition of the enzyme GS using methionine sulphoximine (Msx). The cells, in order to survive, will amplify the DNA encoding GS with concomitant amplification of the DNA encoding the antibody.

Although the cell line used to produce the humanized antibody is preferably a mammalian cell line, any other suitable cell line, such as a bacterial cell line or a yeast cell line, may alternatively be used. In particular, it is envisaged that E. coli-derived bacterial strains could be used. The antibody obtained is checked for functionality. If functionality is lost, it is necessary to return to step (2) and alter the framework of the antibody.

Once expressed, the whole antibodies, their dimers, individual light and heavy chains, or other immunoglobulin forms can be recovered and purified according to standard procedures of the art, including ammonium sulfate precipitation, affinity columns, column chromatography, gel electrophoresis and the like (See, generally, Scopes, R., Protein Purification, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, a humanized antibody may then be used therapeutically or in developing and performing assay procedures, immunofluorescent stainings, and the like (See, generally, Immunological Methods, Vols. I and II, Lefkovits and Pernis, eds., Academic Press, New York, N.Y. (1979 and 1981)).

Studies carried out by Greenwood et al. (1993) have demonstrated that recognition of the Fc region of an antibody by human effector cells can be optimised by engineering the constant region of the immunoglobulin molecule. This could be achieved by fusing the variable region genes of the antibody, with the desired specificity, to human constant region genes encoding immunoglobulin isotypes that have demonstrated effective ADCC in human subjects, for example the IgG1 and IgG3 isotypes (Greenwood and Clark (1993) Protein Engineering of Antibody Molecules for Prophylactic and Therapeutic Applications in Man. Edited by Mike Clark, published by Academic Titles. Section II 85-113). The resulting chimeric or humanized antibodies to mFLC should be particularly effective in inducing ADCC.

Antibodies with fully human variable regions against mFLC can also be prepared by administering the antigen to a transgenic animal which has been modified to produce such antibodies in response to antigenic challenge, but whose endogenous loci have been disabled. Various subsequent manipulations can be performed to obtain either antibodies per se or analogs thereof (see, for example, U.S. Pat. No. 6,075,181).

### Preparation of Genes Encoding Antibodies or Fragments Thereof

Genes encoding antibodies, both light and heavy chain genes or portions thereof, e.g., single chain Fv regions, may be cloned from a hybridoma cell line. They may all be cloned using the same general strategy. Typically, for example, poly(A)⁺mRNA extracted from the hybridoma cells is reverse transcribed using random hexamers as primers. For Fv regions, the V_{H} and V_{L} domains are amplified separately by two polymerase chain reactions (PCR). Heavy chain sequences may be amplified using 5′ end primers which are designed according to the amino-terminal protein sequences of the anti-mFLC heavy chains respectively and 3′ end primers according to consensus immunoglobulin constant region sequences (Kabat et al., Sequences of Proteins of Immunological Interest. 5th edition. U.S. Department of Health and Human Services, Public Health Service, National Institutes of Health, Bethesda, Md. (1991)). Light chain Fv regions are amplified using 5′ end primers designed according to the amino-terminal protein sequences of anti-mFLC light chains and in combination with the primer C-kappa. One of skill in the art would recognize that many suitable primers may be employed to obtain Fv regions.

The PCR products are subcloned into a suitable cloning vector. Clones containing the correct size insert by DNA restriction are identified. The nucleotide sequence of the heavy or light chain coding regions may then be determined from double stranded plasmid DNA using sequencing primers adjacent to the cloning site. Commercially available kits (e.g., the Sequenase™ kit, United States Biochemical Corp., Cleveland, Ohio, USA) may be used to facilitate sequencing the DNA. DNA encoding the Fv regions may be prepared by any suitable method, including, for example, amplification techniques such as PCR and LCR.

Chemical synthesis produces a single stranded oligonucleotide. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, sub-sequences may be cloned and the appropriate subsequences cleaved using appropriate restriction enzymes. The fragments may then be ligated to produce the desired DNA sequence.

Once the Fv variable light and heavy chain DNA is obtained, the sequences may be ligated together, either directly or through a DNA sequence encoding a peptide linker, using techniques well known to those of skill in the art. In one embodiment, heavy and light chain regions are connected by a flexible peptide linker (e.g., (Gly₄Ser)₃) which starts at the carboxyl end of the heavy chain Fv domain and ends at the amino terminus of the light chain Fv domain. The entire sequence encodes the Fv domain in the form of a single-chain antigen binding protein.

### Anti-KMA Antibodies

In one embodiment, the antibody which binds mFLC is an anti-KMA antibody. For example, the antibody may be K121 or a K121-like antibody. K121 is a murine monoclonal antibody (mAb) that specifically recognises human free kappa light chains and an antigen expressed on the surface of kappa-type myeloma cells. This antigen is designated kappa myeloma antigen or KMA (Boux, H A. et al., 1983). It has been established that KMA consists of free kappa light chains expressed in non-covalent association with actin on the cell membrane (Goodnow et al., 1985).

When used herein, the phrase “K121-like antibody” refers to an antibody that comprises the VH region set forth in SEQ ID NO:1 and the VL region set forth in SEQ ID NO:2 or competes with an antibody having the VH region set forth in SEQ ID NO:1 and the VL region set forth in SEQ ID NO:2 for binding to kappa myeloma antigen (KMA).

K121-like antibodies may be identified by their ability to compete with K121 (or chimaeric or humanised forms of K121) in binding to KMA on HMy2 cells. In this procedure, K121 may be conjugated with biotin using established procedures (Hofmann et al., 1982). K121-like antibodies are then evaluated by their capacity to compete with the binding of biotinyolated K121 to KMA on HMy2 cells. The binding of biotinylated K121 to HMy2 cells may be assessed by the addition of fluorescein-labelled streptavidin which will bind to biotin on K121 molecules. Fluorescence staining of cells is then quantified by flow cytometry, and the competitive effect of the K121-like antibody expressed as a percentage of the fluorescence levels obtained in the absence of the competitor.

### Anti-LMA Antibodies

In one embodiment of the invention, the antibody which binds mFLC is an anti-LMA antibody. When used herein the term “LMA” encompasses any free lambda light chain equivalent to a light chain derived from a lambda-type immunoglobulin. The term therefore encompasses a range of lambda light chain polypeptides that can differ in their variable region sequences.

Anti-LMA antibodies will be known to those skilled in the art. For example, antibodies directed against LMA have been used to detect free lambda light chains in serum or urine in tests for diagnosing multiple myeloma (Bradwell et al., 2001). LMA has not been used to date, however, as a target for the treatment of a B-cell mediated immune disorder in a patient.

Examples of suitable anti-LMA antibodies include 3D12 (product #ab1944; AbCam Ltd, Cambridge, UK), CBL317 (Cymbus Biotechnology Ltd, UK), 4G7 (product #ab54380; AbCam Ltd), ME-154 (product #ab9245; AbCam Ltd), 26D (product #CBL317; Chemicon Australia Pty Ltd, Victoria, Australia) or 2G9 (product #CBL109; Chemicon Australia Pty Ltd).

### Cytotoxic Moieties

Suitable cytotoxic moieties for use in the present invention include, but are not limited to, agents such as bacterial or plant toxins, drugs, e.g., cyclophosphamide (CTX; cytoxan), chlorambucil (CHL; leukeran), cisplatin (C is P; CDDP; platinol), busulfan (myleran), melphalan, carmustine (BCNU), streptozotocin, triethylenemelamine (TEM), mitomycin C, and other alkylating agents; methotrexate (MTX), etoposide (VP-16; vepesid), 6-mercaptopurine (6 MP), 6-thioguanine (6TG), cytarabine (Ara-C), 5-fluorouracil (5FU), dacarbazine (DTIC), 2-chlorodeoxyadenosine (2-CdA), and other antimetabolites; antibiotics including actinomycin D, doxorubicin (DXR; adriamycin), daunorubicin (daunomycin), bleomycin, mithramycin as well as other antibiotics; alkaloids such as vincristin (VCR), vinblastine, and the like; as well as other anti-cancer agents including the cytostatic agents glucocorticoids such as dexamethasone (DEX; decadron) and corticosteroids such as prednisone, nucleotide enzyme inhibitors such as hydroxyurea, and the like.

Those skilled in the art will realize that there are numerous other radioisotopes and chemocytotoxic agents that can be coupled to antibodies by well known techniques, and delivered to specifically destroy diseased tissue. See, e.g., U.S. Pat. No. 4,542,225 to Blattler et al. Examples of photo-activated toxins include dihydropyridine- and omega-conotoxin (Schmidt et al., 1991). Examples of imaging and cytotoxic reagents that can be used include ¹²⁵I, ¹³¹I, ¹¹¹In, ¹²³I, ⁹⁹mTc, ³²P, ³H, and ¹⁴C; fluorescent labels such as fluorescein and rhodamine, and chemiluminescers such as luciferin. The antibody can be labeled with such reagents using techniques known in the art. For example, see Wenzel and Meares, Radioimmunoimaging and Radioimmunotherapy, Elsevier, N.Y. (1983) for techniques relating to the radiolabeling of antibodies (see also, Colcher et al., 1986; “Order, Analysis, Results and Future Prospective of the Therapeutic Use of Radiolabeled Antibody in Cancer Therapy”, in Monoclonal Antibodies for Cancer Detection and Therapy, Baldwin et al. (eds), pp. 303-16 (Academic Press 1985)).

In one example, the linker-chelator tiuexutan is conjugated to an antibody which binds mFLC, by a stable thiourea covalent bond to provide a high-affinity chelation site for Indium-111 or Yttrium-90.

When a DNA molecule encoding a cytotoxic agent is present in a therapeutic composition of the invention, the DNA preferably encodes a polypeptide that is a bacterial or plant toxin. These polypeptides include, but are not limited to, polypeptides such as native or modified Pseudomonas exotoxin (PE), diphtheria toxin (DT), ricin, abrin, gelonin, momordin II, bacterial RIPs such as shiga and shiga-like toxin a-chains, luffin (Islam et al., 1990), atrichosanthin (Chow et al., 1990), momordin I (Ho et al., 1991), Mirabilis anti-viral protein (Habuka et al., 1989), pokeweed antiviral protein (Kung et al., 1990), byodin 2 (U.S. Pat. No. 5,597,569), gaporin (Benatti et al., 1989), as well as genetically engineered variants thereof. Native PE and DT are highly toxic compounds that typically bring about death through liver toxicity. Preferably, PE and DT are modified into a form that removes the native targeting component of the toxin, e.g., domain Ia of PE and the B chain of DT. One of skill in the art will appreciate that the invention is not limited to a particular cytotoxic agent.

The term “Pseudomonas exotoxin” (PE) as used herein refers to a full-length native (naturally occurring) PE or a PE that has been modified. Such modifications may include, but are not limited to, elimination of domain Ia, various amino acid deletions in domains II and III, single amino acid substitutions (e.g., replacing Lys with Gln at positions 590 and 606), and the addition of one or more sequences at the carboxyl terminus (see Siegall et al., 1989). Thus, for example, PE38 refers to a truncated Pseudomonas exotoxin composed of amino acids 253-364 and 381-613. The native C-terminus of PE, REDLK (residues 609-613), may be replaced with the sequence KDEL, REDL, and Lys-590 and Lys-606 may be each mutated to Gln.

The term “Diphtheria toxin” (DT) as used herein refers to full length native DT or to a DT that has been modified. Modifications typically include removal of the targeting domain in the B chain and, more specifically, involve truncations of the carboxyl region of the B chain.

### Therapeutic Methods

The methods of the present invention are useful for the treatment or prophylaxis of B-cell mediated immune disorders such as autoimmune disorders, inflammatory disorders and sepsis. The person skilled in the art will understand that B-cell mediated immune disorders do not include B-cell malignancies.

Autoimmune disorders that may be treated with the methods of the present invention include, but are not limited to, rheumatoid arthritis, systemic lupus erythromatosus, diabetes mellitus, multiple sclerosis, Crohn's disease, immune thrombocytopenic purpura, pemphigis vulgaris, autoimmune urticaria, celiac disease, dermatitis herpetiformis, acute rhematic fever, Grave's disease, myasthenic gravis, Sjogren's syndrome, Goodpasture's syndrome, poststreptococcal glomerulonephritis, contact dermatitis, autoimmune thyroiditis, Hashimoto's thyroiditis, Addison's disease, autoimmune haemolytic anaemia, pernicious anaemia, vasculitis caused by anti-neutrophil cytoplasmic antibodies (ANCA), polyarteritis nodosa, autoimmune hepatitis, and primary biliary cirrohsis.

In one aspect, the methods of the present invention utilize the antibodies or binding fragments without modification, relying on the binding of the antibodies or fragments to the surface mFLCs of the plasma cell precursors in situ to stimulate an immune attack thereon. For example, a chimeric antibody, wherein the antigen-binding site is joined to human Fc region, e.g., IgG1, may be used to promote antibody-dependent mediated cytotoxicity or complement-mediated cytotoxicity.

In another aspect of the invention, the therapeutic method may be carried out using mFLC binding moieties, such as an antibody which binds mFLC, to which a cytotoxic agent or biological response modifier is bound. Binding of the resulting conjugate to the plasma cell precursors inhibits the growth of or kills the cells.

As will be appreciated by those skilled in the art, some B-cell mediated immune disorder patients may have significant levels of free lambda light chain in their circulation. As anti-mFLC antibodies react with these free light chains, their presence in the fluid of the subject may reduce the efficiency of the treatment. Accordingly, in one embodiment of the invention the method of treatment further comprises the step of treating the subject to reduce the levels of free lambda light chains circulating in the fluid (e.g. blood) of the subject prior to administration of the anti-mFLC antibody. This additional treatment step may involve, for example, plasmapheresis. As will be known by those skilled in the art, plasmapheresis is a process in which the plasma is removed from blood cells by a device known as a cell separator. The separator works either by spinning the blood at high speed to separate the cells from the fluid or by passing the blood through a membrane with pores so small that only the plasma can pass through. The cells are returned to the subject, while the plasma, which contains the free light chains, is discarded and replaced with other fluids. Medication to keep the blood from clotting (e.g. an anticoagulant) may be given through a vein during the procedure.

It will be appreciated that methods of treating B-cell mediated immune disorders such as, for example, rheumatoid arthritis, systemic lupus erythromatomus, diabetes mellitus, and multiple sclerosis involving the use of anti-mFLC antibodies may be performed in isolation or as an adjunct to other known therapy regimes.

In a further practice of the present invention, anti-mFLC antibodies may be used to remove plasma cell precursors from a patient sample such as bone marrow before reintroduction into the patient. In one non-limiting example, the antibodies are attached to a matrix, such as beads. This may be accomplished by any of several well-known methods for preparing an affinity matrix comprising antibodies or their binding fragments. The patient sample is then exposed to the matrix, such as by passage of the cells over a column containing the matrix, under conditions to promote the binding of the plasma cell precursors in the sample through antigen/antibody interactions with the antibodies or binding fragments attached to the matrix. The plasma cell precursors in the sample adhere to the matrix; while the column effluent, i.e., the non-adherent cellular population, is depleted of plasma cell precursors. The effectiveness of the procedure may be monitored by examining the cells for residual plasma cell precursors, such as by using a detectably-labeled antibody as described below. The procedure may be repeated or modified to increase effectiveness.

### Diagnostic Assays and Kits

Antibodies which bind mFLC are also useful for diagnostic applications, both in vitro and in vivo, for the detection of plasma cell precursors expressing free light chain. In vitro diagnostic methods include immunohistological detection of cells. Immunohistochemical techniques involve staining a biological specimen such as a tissue specimen with an antibody which binds mFLC and then detecting the presence of antibody complexed to its antigen as an antigen-antibody complex. The formation of such antibody-antigen complexes with the specimen indicates the presence of plasma cell precursors expressing free light chain in the tissue. Detection of the antibody on the specimen can be accomplished using techniques known in the art such as immunoenzymatic techniques, e.g., immunoperoxidase staining technique, or the avidin-biotin technique, or immunofluorescence techniques (see, e.g., Ciocca et al., “Immunohistochemical Techniques Using Monoclonal Antibodies”, Methods Enzymol, 121:562-79, 1986 and Kimball, (ed), Introduction to Immunology (2.sub.nd Ed), pp. 113-117 (Macmillan Pub. Co., 1986).

Suitable labels are well known to those of skill in the art. The term “label”, as used herein, refers to a composition detectable by spectroscopic, photochemical, biochemical, immunochemical, or chemical means. For example, useful labels include radioactive molecules such as ³²P, ¹⁴C, ¹²⁵I, ³H, and ³⁵S, fluorescent dyes such as fluorescein or rhodamine, electron-dense reagents, isothiocyanate; chromophores, enzymes (as commonly used in an ELISA), luminescent enzymes such as luciferase and the like.

Such labeled antibodies may be used for the histological localization of the antigens, for ELISA, for cell sorting, and for other immunological techniques to detect and/or quantify the antigens, and cells bearing the antigens, for example. As noted above, a particular use of such labeled antibodies, or fragments thereof, is in determining the effectiveness of plasma cell precursor depletion from bone marrow tissue prior to transplant, particularly autologous bone marrow transplant.

The present invention is also directed to imaging methods for B-cell mediated immune disorders using anti-mFLC antibodies as described hereinabove. The method involves administration or infusion of antibodies as described herein, with or without conjugation to a detectable moiety, such as a radionuclide. After administration or infusion, the antibody, or antibody fragment, binds to the plasma cell precursors, after which the location of the antibodies is detected. For detectably-labeled antibodies, such as those labeled with a radionuclide, imaging instrumentation may be used to identify the location of the agent within the body. For use of unlabeled antibodies, a second, detectable reagent may be administered which locates the antibodies, and thus may be suitably detected. These methods have been used for other antibodies, and the skilled artisan will be amply aware of these various methods for imaging the location of antibodies or fragments within the body.

This invention also embraces kits for research or diagnostic purposes. A kit typically includes one or more containers containing an anti-mFLC antibody. The anti-mFLC antibody may be derivatized with a label or, alternatively, it may be bound with a secondary label to provide subsequent detection. As described above, such labels may include radiolabels, fluorescent labels, enzymatic labels, i.e., horseradish peroxidase (HRP), or the like. The kit may also contain appropriate secondary labels (e.g., a sheep antimouse-HRP, or the like). The kit may also contain various reagents to facilitate the binding of the fusion polypeptides, the removal of non-specific binding antibodies, and the detection of the bound labels. Such reagents are well known to those of skill in the art.

In a further aspect of the present invention, compositions are provided which comprise the anti-mFLC antibody bound to a solid support. A solid support for use in the present invention will be inert to the reaction conditions for binding. A solid phase support for use in the present invention must have reactive groups or activated groups in order to attach the monoclonal antibody or its binding partner thereto. In another embodiment, the solid phase support may be a useful chromatographic support, such as the carbohydrate polymers SEPHAROSE®, SEPHADEX®, or agarose. As used herein, a solid phase support is not limited to a specific type of support. Rather, a large number of supports are available and are known to one of ordinary skill in the art. Solid phase supports include, for example, silica gels, resins, derivatized plastic films, glass beads, cotton, plastic beads, alumina gels, magnetic beads, membranes (including, but not limited to, nitrocellulose, cellulose, nylon, and glass wool), plastic and glass dishes or wells, etc.

Methods for using the research and diagnostic kits described above are generally well known, and are generally provided in an instruction manual for use of the kit.

### EXAMPLES

### Example 1

### In Vitro Activation of Normal Human Peripheral Blood B-Cells

CD19+ peripheral blood derived B cells were activated in vitro using two different protocols, 1. a combination of IL-21, anti-CD40 and anti-IgM, or 2. formalin fixed Staphylococcus aureus bacteria (SAC).

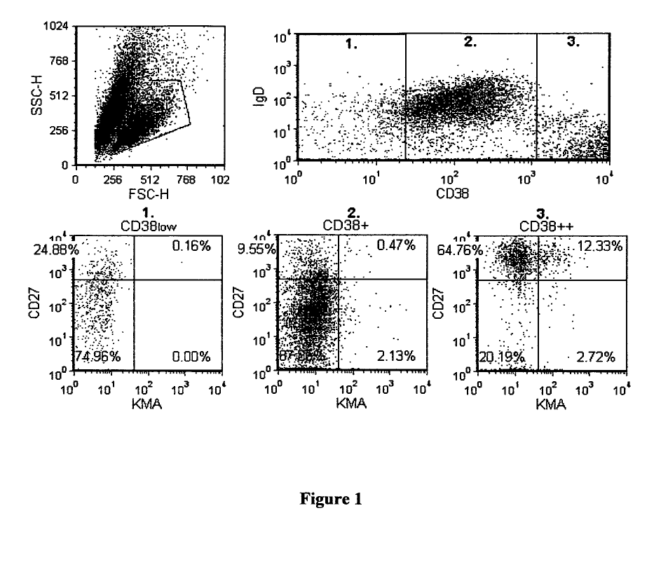

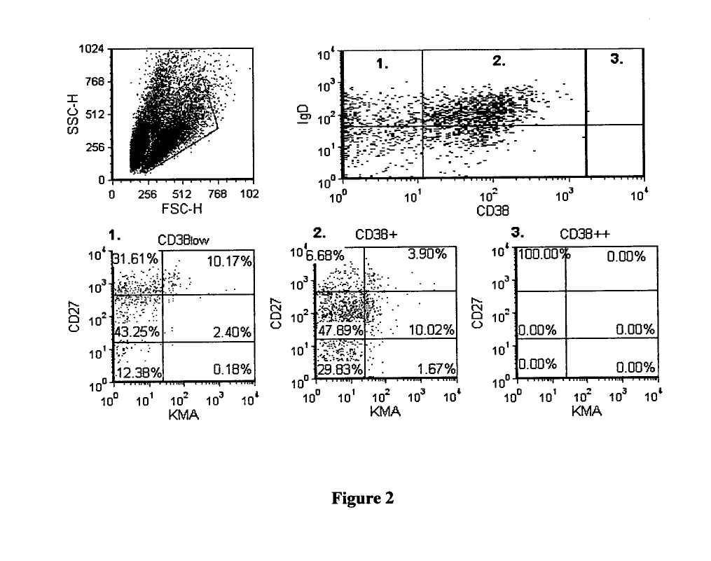

Activation of the purified B cells with IL-21, anti-CD40 and anti-IgM generated plasma cells, including some that expressed KMA on their surface (KMA being defined by mKap [K121] reactivity). Phenotypic analysis of the KMA expressing cells by flow cytometry revealed them to be a subset of plasma cells as defined by surface IgD−, CD27++ and CD38++(FIG. 1). These KMA+/IgD−/CD27++/CD38++ represented approximately 12% of all CD38++ B cells. In contrast, stimulation with SAC failed to produce any plasma cells. However KMA expression was found in two distinct populations; CD27++ and CD38low plasmablasts (approximately 10% of CD38low population) as well as CD27+/++ and CD38+ plasmablasts (approximately 14% of CD38+ population; FIG. 2).

### Example 2

### In Vivo Expression of KMA in Normal Human Lymphoid B Cells

Given the results obtained with the in vitro activation system, the inventors determined whether similar types of cells express KMA in vivo. Mononuclear cells from human tonsils were analyzed by FACS for KMA expression along with the B cell markers CD19, CD38 and CD45.

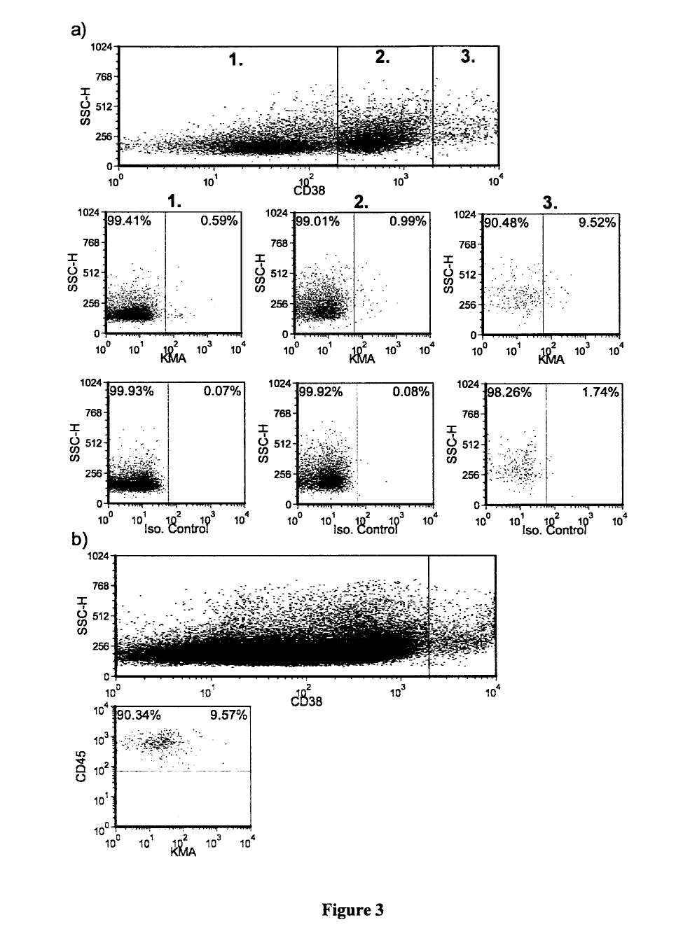

The inventors found a small population of KMA expressing cells in the CD38++ plasma cell fraction (approximately 7.8% above isotype control; FIG. 3a, Gate 3.). Interestingly, these cells also expressed CD45, which is representative of an immature plasma cell phenotype (FIG. 3b).

In addition to flow cytometric evaluation of KMA expressing cells, an immunohistochemical (IHC) study was performed on normal tissue. Cryopreserved, unfixed tissue sections from normal donors were stained with FITC labeled MDX1097 (a human-mouse chimeric antibody comprising of the mKap variable domains) followed by mouse anti-FITC secondary and goat anti-mouse peroxidase. Slides were treated with chromagen substrate and analyzed for expression.

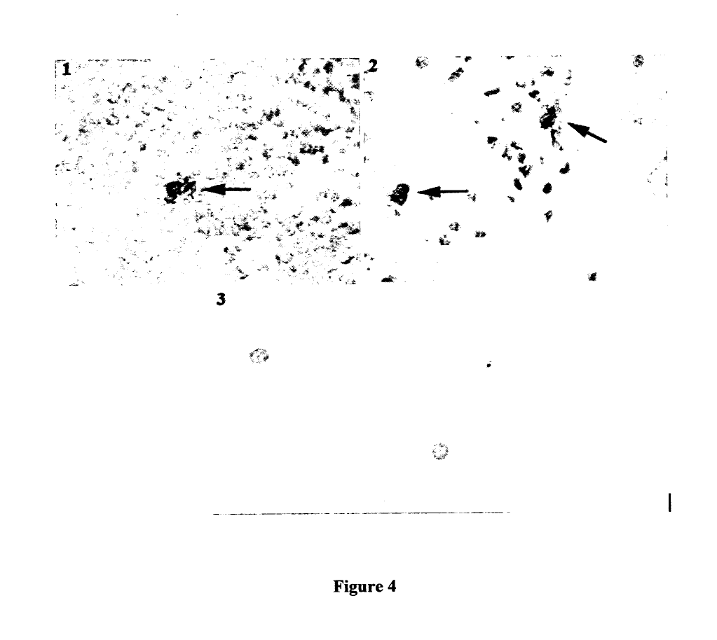

IHC staining of normal human tissue with MDX1097 revealed a small number of KMA positive cells with plasma cell and plasmablast morphology (ie. cells with large cytoplasm and round nuclei) in tonsil and salivary gland. Staining was observed both on the membranes as KMA and intracellularly, indicating these cells contain a large pool of free κLC. No KMA expressing cells were observed in normal peripheral blood, confirming previous results (FIG. 4; Walker et al., 1985).

### Example 3

### KMA and LMA Expression on Activated Peripheral Blood B Cells

The CD19+ peripheral blood B cells were isolated from donor samples obtained from the Australian Red Cross. Following density gradient centrifugation for isolation of peripheral blood mononuclear cells (PBMCs), CD19+ cells were purified using magnetic MicroBeads and the MACS separation system or the autoMACS Pro instrument (Miltenyi Biotec, Germany). The CD19+ cells were activated by incubating in media containing IL-21 (1 mg/mL; Invitrogen, USA), anti-CD40 antibody (10 μg/mL; R & D Systems, USA) and anti-IgM antibody (50 μg/mL; Sigma-Aldrich, USA) antibodies at 37° C., 5% CO₂ for 6-7 days

The activation of CD19+ peripheral blood B cells with IL-21, anti-CD40 and anti-IgM, resulted in the generation of KMA+ and LMA+ cells. The phenotype of the KMA and LMA expressing cells was CD27++, CD38++, CD45+ and IgD−, thus confirming previous results showing KMA expression on a subset of late plasmablasts (CD45 expression was not previously investigated).

In this study KMA was observed in 6-60% of the CD38++/CD27++/CD45+ population, which is supported by the previous evidence showing KMA expression on approximately 14% of a subset of activated CD19+ B cells. The expression of LMA was not previously investigated, and we wished to examine the presence/absence of LMA on in vitro activated CD19+ cells. This study demonstrated that 10-20% of CD27++, CD38++, CD45++ and IgD− cells expressed LMA. A summary of the results obtained is provided in Table 1.

TABLE 1
Summary of KMA and LMA expression on activated plasmablasts.

| Donor | Day post-activation | KMA Expression | LMA expression |
|---|---|---|---|
| 1 | 6 | 60.00% | 20.80% |
| 2 | 6 | 0.00%* | 10.00% |
| 3 | 7 | 12.54% | 12.52% |
| 4 | 6 | 6.12% | 13.73% |
| 5** | 6 | 9.09% | 0.00% |

Following activation of peripheral B cells in the presence of IL-21, anti-CD40 and anti-IgM antibodies, KMA and LMA expression was investigated. KMA and LMA were detected on CD27++, CD38++, CD45+ and IgD− cells in percentages shown above. The MACS separation system was used for donors 1-3, while the autoMACS Pro instrument was used for donors 4-5.
*low number of events collected in the flow cytometry run
**low viability

### Discussion

Phenotypic and morphological analysis has demonstrated that KMA and LMA are expressed on a subset of normal plasmablasts and immature plasma cells as mFLC. These cell types represent a functionally unique subpopulation of antibody-secreting cells (Shapiro-Shelef et al., 2005).

During a normal immune response, much of the earlier phase of antibody production is dominated by plasmablasts and immature plasma cells. Eventually these cells terminally differentiate into mature plasma cells that either undergo rapid cell death or migrate to the bone marrow and persist in longevity due to survival cues from the bone marrow microenvironment (Manz et al., 1997).

Many studies have analyzed the role of plasmablasts and plasma cells in various autoimmune pathologies. Since plasmablasts and immature plasma cells are both a source of autoantibodies, as well as the precursors of mature plasma cells, they are considered as some of the main effector cell types in numerous autoimmune diseases. For example, plasmablast and immature plasma cell involvement has been shown in multiple sclerosis, rheumatoid arthritis, systemic lupus erythematosus, Sjogren's syndrome, diabetes and reactive plasmacytosis (Cepok et al., 2005; William et al., 2005; and Jego et al., 1999). Given that KMA and LMA are expressed specifically on these cell types as mFLC, KMA and LMA represent attractive therapeutic targets for the treatment of various autoimmune states.

### Example 4

### Direct Cellular Cytotoxicity Mediated by Antibodies that Bind mFLC

4.1 Proliferation Assays

Cells will be incubated with various concentrations of an antibody that binds mFLC, in medium and conditions suitable for cell growth. By way of example, an antibody may be incubated with cells in RPMI 1640 medium supplemented with 5% fetal calf serum at 37° C. for various lengths of time between 1 hour and 4 days. The proliferative state of the antibody treated cells, in comparison to cells alone, will be measured by using reagents that measure the metabolic health of the cell population. One such reagent is the MTS solution (Promega, USA) that is converted to formazan by metabolically active cells. This conversion is associated by generation of absorbance at 490 nm which can be measured using an absorbance reading instrument.

4.2 Apoptosis Assays

Cells will be incubated with various concentrations of the antibody, in medium and conditions suitable for cell growth. For example, cells will be incubated with various concentration of the antibody in RPMI 1640 medium supplemented with 5% fetal calf serum at 37° C. for 4 hours. The apoptotic state of the antibody treated cells, in comparison to cells alone, will be investigated using Annexin-V-FITC (fluorescein isothiocyanate) or propidium Iodide (PI) reagents and flow cytometry. Annexin-V binds to the negatively charged phospholipid phosphatidylserine (PS) that is redistributed from the inner to the outer leaflet of the cell membrane in the early stages of apoptosis. Cells that are necrotic will be detected by PI. Flow cytometry enables the measurement of PI and FITC fluorescence, thus, enabling the distinguishing of apoptotic and necrotic cells.

4.3 Antibody Cross-Linking

Cells will be incubated with various concentrations of the antibody, and a cross-linking reagent, in medium and conditions suitable for cell growth such as described above. The cross-linking reagent will be an antibody preparation that binds to the antibody used to target cells. For example, if the anti-KMA or anti-LMA antibody is a human IgG1, the cross-linking antibody will be a polyclonal preparation specific for human IgG1. Antibody cross-linking is known to potentiate the direct cytotoxic effects of antibodies. The effects of antibody cross-linking on the target cell proliferation will be measured using the proliferation and apoptosis assay methodologies described above.

### Example 5

### Cell-Mediated Cellular Cytotoxicity by Antibodies that Bind mFLC

### 5.1 Antibody Dependent Cellular Cytotoxicity (ADCC)

Effector cells for use in ADCC assays will be either peripheral blood mononuclear cell (PBMC) preparations or specific cell populations such as natural killer (NK) cells or monocytes contained within PMBC preparations. PBMCs will be isolated from blood using Ficoll density gradients. Blood will be overlaid on Ficoll, the gradient will be centrifuged and PBMCs will be collected from the interface of the gradient.

Specific cell populations will be isolated from PBMC preparations generated as above using magnetically labeled antibody preparations (Miltenyi Biotec, Germany) to deplete undesired cells. For example, NK cells will be isolated using magnetically labeled antibody cocktails which deplete non-NK cells from PMBC preparations. The magnetically labeled cells will be retained (AutoMacs Pro instrument, Miltenyi Biotec, Germany) and NK cells will be collected.

In ADCC assays, the effector and target cells, coated with various concentrations of antibody, will be mixed in various effector:target cell ratios and the mixture will be incubated in a suitable medium and under conditions suitable for cell growth. For example, the mixture may be incubated in RPMI supplemented with 10% fetal calf serum at 37° C. for 16 hours. At the end point of the assay, the degree of cell lysis will be measured. For example, the level of intracellular lactate dehydrogenas (LDH) released will be measured as an indicator of cell lysis using the CytoTox-ONE Homogenous Membrane Integrity Assay Kit (Promega, USA).

5.2 Antibody Dependent Cellular Phagocytosis (ADCP)

In ADCD assay the phagocytic effector cells will be generated in vitro. PMBC preparations will be used to isolate monocytes by depleting non-monocytic cells via the use of magnetically labeled antibody cocktails and the AutoMacs Pro instrument (Miltenyi Biotec, Germany). Purified monocytes will be culture in vitro in medium suitable for culturing and differentiation of monocytes to macrophages.

Macrophages will be plated out in culture chambers supporting their adhesion and suitable for the study of cellular interactions using a confocal microscope. Target cells will be stained with a fluorescent dye such as pHRodo (Invitrogen, USA), which is pH sensitive, coated with antibody at various concentrations and placed in the chambers containing macrophages. The pHRodo dye's fluorescent spectra changes once it has been exposed to acidic pHs. The cell mixture will be incubated under suitable conditions. For example, the cell mixture may be incubated in RPMI 1640 supplemented with 10% fetal calf serum for 2 hours at 37° C. At the end point of the assay, unbound cells will be washed and the macrophages will be stained with a fluorescently conjugated antibody specific for a marker exclusively expressed on the surface of macrophages. Finally, the cells will be analyzed using a confocal microscope.

Macrophages will be identified via the fluorescence resulting from the specific labeling of their cellular membrane. Macrophages that have ingested target cells will have, in addition to the staining of their membranes, the fluorescence imparted by the pHRodo dye within the acidic endosomes of the cell. Phagocytosis will be measured, by counting in a certain number of microscopic field of views, the total number of cells and the number of phagocytic cells.

5.3 Complement Dependent Cytotoxicity (CDC)

Target cells will be incubated in the presence of complement (either purified or human serum containing complement) and antibody under conditions supporting cell growth. For example, the target cells may be incubated in the presence of complement and antibody in RPMI supplemented with 10% fetal calf serum for between 30 minutes to 12 hours at 37° C. At the end point of the assay, the degree of cell lysis or the metabolic state of cells (reflecting cell growth) will be measured. Cell lysis will be measured using the method described in section 5.1 above. The metabolic state of cells will be measured using reagents such as Alamar Blue (Invitrogen, USA). After the addition of Alamar Blue, the fluorescence detected in the complement and antibody treated cell mixtures is proportional to the number of viable cells.

All publications discussed and/or referenced herein are incorporated herein in their entirety.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Benatti et al. (1989) Eur J Biochem, 183: 465-470.
Beychok (1979) Cells of Immunoglobulin Synthesis, Academic Press, New York, p 69.
Bird et al. (1988) Science, 242:423-426.
Boux, H A. et al. (1983) J Exp Med. 158: 1769.
Bradwell et al. (2001) Clin. Chem. 47: 673-680.
Browning (2006) Nat Rev Drug Discovery, 5: 564-576.
Cepok et al. (2005) Brain, 128: 1667.
Chow et al. (1990) J Biol Chem, 265: 8670-8674.
Edwards and Cambridge (2006) Nat Rev Immunol, 6: 394-403.
Goodnow et al. (1985) J. Immunol. 135: 1276
Greenwood et al. (1993) Eur J Immunol, 23: 1098-1104.
Habuka et al. (1989) J Biol Chem, 264: 6629-6637.
Ho et al. (1991) BBA, 1088: 311-314.
Hofmann K, et al. (1982) Biochemistry 21: 978-84.
Huston et al. (1988) Proc Natl Acad. Sci. USA, 85:5879-5883.
Islam et al. (1990) Agricultural Biological Chem, 54: 1343-1345.
Jego et al. (1999) Blood, 94: 701.
Jones et al. (1986) Nature, 321, 522-525.
Kung et al. (1990) Agric Biol Chem, 54: 3301-3318.
Manz et al. (1997) Nature, 388: 133-134.
Morrison et al. (1984) Proc Natl Acad Sci USA, 81:6851-6855.
Schmidt et al. (1991) J Biol. Chem. 266:18025-33.
Shapiro-Shelef et al. (2005) Nature Reviews Immunology, 5: 230-242.
Siegall et al. (1989) J Biol Chem 264: 14256-14261.
Sun et al. (1986) Hybridoma 5 Suppl 1:517-20.
Urlaub and Chasin (1980) Proc Natl Acad Sci USA. 77:4216-4220.
Walker et al. (1985) Adv Exp Med Biol, 186: 833-841.
William et al. (2005). J Immunol, 174: 6879-6887.

1. A method for the treatment or prophylaxis of a B-cell mediated immune disorder in a subject, the method comprising administering to the subject an effective amount of an antibody which binds membrane-bound free light chain (mFLC).

2. The method of claim 1, wherein the antibody binds mFLC on a plasma cell precursor.

3. The method of claim 2, wherein the antibody is conjugated to a cytotoxic moiety or biological response modifier.

4. The method according to claim 3, wherein the cytotoxic moiety is a toxin, a chemotherapeutic agent, or a radioactive agent.

5. The method according to claim 3, wherein the cytotoxic moiety is a nucleic acid molecule encoding a cytotoxic polypeptide.

6. The method according to claim 3, wherein the biological response modifier is a lymphokine, a cytokine or an interferon.

7. (canceled)

8. The method according to claim 1, wherein the antibody binds KMA.

9. The method according to claim 8, wherein the antibody is a K121-like antibody.

10. The method according to claim 9, wherein the antibody comprises the VH region set forth in SEQ ID NO:1 and the VL region set forth in SEQ ID NO:2 or competes with an antibody having the VH region set forth in SEQ ID NO:1 and the VL region set forth in SEQ ID NO:2 for binding to kappa myeloma antigen (KMA).

11. The method according to claim 1, wherein the antibody binds LMA.

12. The method according to claim 1, wherein the B-cell mediated immune disorder is an autoimmune disease.

13. The method according to claim 12, wherein the autoimmune disease is selected from rheumatoid arthritis, systemic lupus erythromatomus, diabetes mellitus, and multiple sclerosis.

14. A method of inhibiting the growth of or killing plasma cell precursors in a subject, the method comprising administering to the subject an effective amount of an antibody which binds mFLC.

15. The method of claim 14, wherein the antibody is conjugated to a cytotoxic moiety or biological response modifier.

16. The method according to claim 15, wherein the cytotoxic moiety is a toxin, a chemotherapeutic agent, or a radioactive agent.

17. The method according to claim 15, wherein the cytotoxic moiety is a nucleic acid molecule encoding a cytotoxic polypeptide.

18. The method according to claim 15, wherein the biological response modifier is a lymphokine, a cytokine or an interferon.

19. A method for localizing plasma cell precursors in a subject, the method comprising administering to the subject an antibody which binds mFLC, allowing the antibody to bind to cells within the subject, and determining the location of the antibody within the subject.

20. The method according to claim 19, wherein the antibody is detectably labeled.

21. The method according to claim 1, wherein the antibody which binds mFLC is a chimeric antibody or a humanised antibody.

22.-25. (canceled)
